(19) United States
(12) Patent Application Publication
(10) Pub. No.: US 2020/0147290 A1
(43) Pub. Date: May 14, 2020

(54) LIMB STABILISATION APPARATUS AND METHODS

(71) Applicant: University of Strathclyde, Glasgow (GB)

(72) Inventor: Terence Gourlay, Glasgow (GB)

(21) Appl. No.: 16/612,829

(22) PCT Filed: May 9, 2018
(86) PCT No.: PCT/GB2018/051239

§ 371 Date: Nov. 12, 2019

(30) Foreign Application Priority Data
May 12, 2017 (GB) 17076149.8

(51) Int. Cl. A61M 1/36

(52) U.S. Cl. CPC A61M 1/3613; A61M 2210/08; A61M 1/3659; A61M 1/3639

(57) ABSTRACT

A limb-stabilisation apparatus (100) comprises a limb-receiving container (110) arranged to receive a portion of the limb (5) and to collect blood exited from the limb; and a blood recirculation system (120) configured to recirculate blood from the limb-receiving container (110) to one or more regions of the limb. Advantageously, the limb-stabilisation apparatus (100) may further comprise or may be associated with a limb-compressing apparatus (210) configured to reduce, restrict or prevent blood flow in the region of the limb-compressing apparatus.

### FIELD OF INVENTION

The present invention relates to an apparatus, systems and methods for preserving a limb of a subject. In particular, though not exclusively, the present invention relates to apparatuses and methods for providing circulatory and/or metabolic support to an injured limb.

### BACKGROUND TO INVENTION

Salvage and/or recovery of a person's injured extremities or limbs is an important area of medical research. One common environment leading to injuries to a person's extremities or limbs in the military environment. Combat casualty mortality, defined as individuals who were killed in action and those who subsequently succumbed to wounds, is mainly the result of vascular trauma and haemorrhage (Kelly et al, 2008). However, approximately 25% of military personnel who die from explosion or gunshots have potentially survivable injuries (Eastridge et al, 2013) and thus many casualties rely on effective prehospital trauma care.

Data on potentially survivable deaths from the Vietnam War showed that 60% were from extremity haemorrhage exsanguination, 33% from tension pneumothorax, and 7% from airway obstruction (Maughon, 1970; Holcomb et al 2007; Kelly et al 2008; Bellamy 1984). Since then, improvements in prehospital care have taken precedence in military medicine to improve survival from combat injury. As such, battlefield survival of US casualties has significantly improved, in that a greater percentage of wounded soldiers survived during Operations Enduring and Iraqi Freedom (OEF and OIF) than in any other previous conflict (Starnes et al, 2006). This is due to lifesaving technologies such as improved body armour and improved patient resuscitation (Kragh et al, 2009; Ennis et al, 2008; Holcomb et al 2008; Eastridge et al 2006). As a result, survivable injury severity has significantly increased, and data collected from 2003 to 2006 in OIF indicate that the annual proportion of severe extremity injuries of US casualties rose from 21% to 44% (Kelly et al 2008).

Uncontrolled external haemorrhage of the extremities (also known as compressible bleeding) still remains the primary cause of preventable deaths in the field (Heldenberg et al 2015). Mortality rates of up to 9% have been recorded as being a result of poorly treated external haemorrhage (Starnes et al 2006; Mabry et al, 2000). During OIF, extremity vascular injuries accounted for 50-70% of all injuries, and exsanguination from these injuries is the leading cause of preventable death on the modern battlefield (Starnes et al 2006).

British and US Army battlefield analysis has shown that approximately 50% of injuries were to the extremities during OIF (Belmont et al 2010; Ramalingam 2004). Improvements in personal protective equipment (PPE) have considerably increased likelihood of survival in Iraq and Afghanistan (Nelson et al 2008; Galarneau et al 2008). Technological advances include body armour with blast resistant ballistic goggles or glasses worn with improved Kevlar Advanced Combat Helmets, although these do not protect the extremities (Hildreth, 2009; Nyein et al, 2010) in common contemporary situations where blast forces are a huge risk to the vulnerable lower extremities from detonated ground level IEDs, or where they are directed upward through the floor of a vehicle (Fox et al, 2005). Low intensity conflict in an urban environment is a modern battlefield phenomenon (as in OEF and OIF) which exposes soldiers to urban warfare and against weapons such as grenades, mines and IEDs (Starnes et al 2006; Husum et al 2004). This environment, coupled with personal armour that focuses on torso, head and neck protection, is a dominant cause of the sharp increase in the proportion of extremity injuries (Gofrit et al 2005; Gondusky & Reiter, 2005).

For allied military personnel in the Iraq and Afghanistan conflicts, explosive devices were the mechanism of injury associated with most (87.9%) amputations (Stansbury et al 2008). In Iraq alone, 1,158 US military personnel suffered limb amputations (Fischer, 2010). In the Iraq and Afghanistan wars, a military tactic from opposition forces was the placement of IEDs which increased the incidence of ‘dismounted IED injuries’—these leg and genital wounds resulted in approximately 6% of seriously wounded soldiers receiving lower limb amputations in the first 7 years. The rate of blast injuries has increased in both US and UK casualties since 2010 in Afghanistan and often involved multiple limb loss (Wallace, 2012) and the constellation of lower limb amputations has been dubbed the ‘new signature wound of the war’ (Brown, 2011), a term previously used in relation to mild traumatic brain injury (Okie, 2005).

In Afghanistan 2010, the rate of amputations increased whilst US combat deaths declined, from 437 to 368, and this further confirmed improvements in wound survivability (Carroll, 2012). Furthermore, in 2010-11, nearly half a sample of US combat fatalities suffered bilateral lower limb extremity amputations, with almost a third losing three limbs (Dismounted Complex Blast Injury Task Force, 2011). The socioeconomical impact of extremity injuries from OEF and OIF was considerable, in which this subset of casualties accounted for almost 65 percent of inpatient care (responsible for the most operating room time use, the largest the number of surgeries performed and the number of occupied beds (Cho et al 2005)), caused the greatest number of disabled soldiers, and resulted in the greatest projected disability costs (Masini et al 2009).

One well-known means of controlling potentially life-threatening extremity haemorrhage exsanguination, particularly in situations where the subject is not in proximity to any medical facilities, the use of tourniquets. However, while isolation of an injured limb may reduce life-threatening extremity haemorrhage exsanguination, application of a tourniquet isolates the limb. This ischemic isolation of the subject's limb, particularly if sustained for a prolonged period of time, may cause irreversible damage to the limb and may reduce the likelihood of subsequently full or partial recovery of the limb, which may thus necessitate amputation of the isolated limb. There currently exists no convenient, portable and cost effective means of isolating a limb and maintaining circulatory and/or metabolic needs of the isolated limb which can be quickly and easily deployed and applied to a subject.

It is an object of at least one embodiment of at least one aspect of the present invention to alleviate and/or mitigate one or more problems or disadvantages associated with the prior art.

### SUMMARY OF INVENTION

According to a first aspect of the present invention there is provided a limb-stabilisation apparatus comprising:

a limb-receiving container arranged to receive a portion of the limb and to collect blood exited from the limb; and

a blood recirculation system configured to recirculate blood from the limb-receiving container to one or more regions of the limb.

Advantageously, the limb-stabilisation apparatus further comprises or may be associated with a limb-compressing apparatus configured to reduce, restrict or prevent blood flow in the region of the limb-compressing apparatus. This may assist in isolating the injured limb from the subject's circulatory system, and may help prevent or reduce bleeding and/or extremity haemorrhage exsanguination. The limb-compressing apparatus may be termed a tourniquet.

Typically, the limb-receiving container may be configured to receive a portion of the limb below the limb-compressing apparatus. The term “below” will not be construed as relating to any geometrical arrangement, but rather as referring to a location relative to the body's circulatory system. As such, the term “below” will herein be understood to refer to a region nearer a limb's extremity or to a region of the limb further away from the heart relative to the limb-compressing apparatus.

Advantageously, the limb-receiving container may be configured to receive and/or contain blood. By such provision, the limb-receiving container may be capable of containing the blood exited from the injured limb, thus ensuring that all the blood lost by the subject may be recirculated. The limb-receiving container may be impermeable to liquids, e.g. blood. The limb-receiving container, e.g. at least a lower portion thereof and/or a portion of the container configured to receive blood, may be made from a durable and/or hygienic material such as a plastic material, e.g. polycarbonate, acrylic, polyethylene, polypropylene, polyester, polyurethane, PVC, or the like.

The limb-receiving container may comprise and/or may define a sump for collecting blood exited from the limb and/or contained in the limb-receiving container.

The apparatus, e.g. blood recirculation system, may comprise a pump for pumping and/or circulating blood.

The pump may be in fluid communication with the sump. The apparatus may comprise a sump outlet. The sump outlet may provide fluid communication between the sump and the pump, e.g., via a sump connector.

The pump may be capable of recirculating blood to one or more regions of the limb. In an embodiment, the pump may be connectable to one or more cannulae which can each be connected to a region of the limb. Typically, the/each cannula may be connected to an arterial vessel of the limb. By such provision, the apparatus may allow recirculation of the blood lost by the limb and collected into the sump back into an arterial system of the limb, thus maintaining blood flow through the limb in a simple and effective manner, for example prior to, or after, surgery. The blood recirculation system may comprise the one or more canulae.

In an embodiment, there may be provided a plurality of cannulations, each cannulation being connected to an arterial vessel upstream from an associated wound, e.g., upstream from a wound from which blood exits the vessel.

In an embodiment, the/a pump may be connectable to or may be associated with one or more cannulae, e.g. may be connectable to or may be associated with a plurality of cannulae. There may be provided a cannulation router, e.g. associated with a/the pump, and which may be configured to guide pumped blood to one or more cannulae. For example, the cannulation router may have at least one inlet, and a plurality of outlets, each outlet having an open and a closed position and being connectable to a respective cannula.

In another embodiment, each cannula may be connectable to or associated with an associated pump. There may be provided a plurality of pumps, each pump being connectable to or associated with a respective cannula.

The apparatus, e.g. the limb-receiving container, may comprise or may be provided with or may be attachable to a lid. The lid may be detachable from the container. Alternatively, or additionally, the lib may be openable so as to allow a limb to be placed in the container. In an embodiment the lid may be hingedly attached to the container. The provision of a lid may allow, in use, protection of the limb and/or may provide a hygienic and/or sealed environment for a/the injured limb.

The container, e.g. lid, may have one or more openings such as holes or apertures configured to receive a medical tube such as a cannula. There may be provided a plurality of openings located at different positions in the container and/or lid. By such provision, a/the cannula may be easily connected to a vessel, e.g. an arterial vessel, on the limb at a location near a particular opening, in use.

Advantageously, at least a portion of the lid may be made of a transparent material. By such provision, a used may be able to connect a cannula to an arterial vessel and/or may be able to monitor the apparatus and/or the subject's limb, in use. The lid may be made from a durable and/or hygienic material such as a plastic material. Typically, the lid may be made from a rigid material, which may help provide support and/or protection for a/the injured limb. The lid may be made from polycarbonate, acrylic, polyethylene, polypropylene, polyester, polyurethane, PVC, or the like.

The container may have or may be provided with a limb-suspension member configured to suspend the limb, in use. Advantageously the limb-suspension member may allow the limb to be supported within the container, while ensuring the limb does not rest on or does not contact a substantial area of the container. This may provide a more hygienic environment, allowing the blood to exit vessels and/or wounds in the limb and be collected in the sump without significant or prolonged contact with an outer surface of the limb, e.g. skin thereof, thus reducing the risks of infection.

The limb-suspension member may comprise or may be provided in the form of a net or mesh. A net of mesh may provide adequate support to the limb, while helping blood drainage and/or collection into the container, e.g. blood sump.

In use, the apparatus may be configured or inclined such that the blood lost from the limb flows and/or is collected into the sump. Typically, the apparatus, e.g. limb-receiving container, may be arranged such that the blood sump is located near a lower end of the container and/or may be inclined downwards towards the blood sump.

The blood sump may be provided with blood measuring means for measuring the level and/or volume of blood in the sump. The blood measuring means may comprise or may be provided in the form of graduations or markings on the blood sump. Alternatively or additionally, the blood measuring means may comprise a sensor configured to measure the level and/or volume of blood in the sump.

The apparatus may comprise at least one blood pressure sensor for measuring the pressure within the blood recirculation system. At least one blood pressure sensor may be associated with a/the pump(s). At least one blood sensor pressure may be associated with or may be located near a/the cannula. At least one blood sensor pressure may be associated with or may be located near a/the sump connector.

By such provision, during operation of the blood recirculation system, the blood pressure within the pressure blood recirculation system may be monitored.

The pump may be controllable. The pump, e.g. pump power, may be controllable and/or adjustable based on the measured blood pressure and/or blood level.

If the blood pressure within the pressure blood recirculation system falls below a predetermined or desired value, the pump power may be altered, for example in order to reach the predetermined or desired pressure.

If the blood level and/or volume in the sump falls below a predetermined level, blood recirculation may be stopped, for example a/the associated pump may be stopped, e.g., until blood level in the sump reaches a/the predetermined level

Alternatively, or additionally, if the blood level and/or volume in the sump falls below a predetermined level, external blood may be supplied or fed, e.g. automatically, into the recirculation system. There may be provided an external blood container, e.g. blood bag, containing external blood.

By such provision, the apparatus/system ensures that, in the event that there are insufficient blood levels in the sump to provide the desired blood pressure and/or blood flow, the recirculation system does not cause air to be pumped into the subject.

The apparatus may comprise or may be associated with a blood oxygenating device.

The apparatus may comprise or may be associated with a blood temperature control device, e.g. cooling device.

Advantageously, the apparatus may comprise or may be associated with a combined and/or integrated blood oxygenating device and temperature control device. By such provision, the device may provide not only blood recirculation to the injured limb, but may also provide blood oxygenation thus providing the metabolic needs of the limb. The provision of a temperature control device, e.g. a cooling device, capability may also help reduce the oxygen requirements of the limb.

Conveniently, the temperature control device may comprise a plurality of solid state heating and cooling elements, e.g. Peltier elements. This may improve the portability of the device.

The combined and/or integrated blood oxygenating device and temperature control device may further comprise a pump. The pump may comprise or may be the pump of the apparatus used for pumping and/or circulating blood.

In an embodiment, there may be provided a combined or integrated pumping, blood oxygenating device and temperature control device, for example as described in PCT Application Publication No. WO 2012/013925 (Gourlay), the content of which is incorporated herein by reference.

The limb-stabilisation apparatus, e.g. blood recirculation system thereof, may comprise a blood-treatment unit. The blood treatment unit may be configured to alter, e.g. to remove, certain components of the blood. In an embodiment, the blood treatment unit may be configured to remove one or more components of the blood that are susceptible of inducing or suppressing an inflammatory response in the subject. The blood treatment unit may comprise a filtration element. The blood treatment unit may comprise an element, e.g. a filtration element, capable of removing cytokines.

The limb-stabilisation apparatus, e.g. blood recirculation system thereof, may comprise a blood-sterilisation unit. The blood sterilisation unit may be configured to remove or kill pathogenic components in the blood. The blood sterilisation unit may be a light sterilisation unit, which may be configured to emit or pass radiation through the blood, e.g. UV radiation, or a High Intensity Narrow Spectrum (HINS) light.

The limb-compression apparatus may be or may comprise a tourniquet. A tourniquet may be defined as an apparatus capable of compressing a limb of a subject, for example in order to reduce, restrict or preventing blood flow in the region of the tourniquet. By such provision, in the event of an injury, the use of a tourniquet may reduce or may prevent bleeding though a wound and/or may reduce or may prevent external haemorrhage of the injured limb.

The limb-compressing apparatus may comprise at least two expandable members configured for applying pressure on a limb of a subject; and actuating means for actuating the at least two expandable members. Advantageously, the actuating means may be configured to actuate each expandable member independently and/or selectively.

Typically, the apparatus may have two, e.g., a pair of, expandable members.

The apparatus may comprise a housing. The expandable members may be provided or may be located at least partially within the housing.

In use, the housing may define an opening configured for receiving a limb of a subject, e.g. a portion of an arm or a leg. In use, the housing may contact the limb of the subject.

In use, at least one expandable member, typically each expandable member, may define an opening configured for receiving a limb of a subject, e.g. a portion of an arm or a leg.

The expandable members may be spaced apart, e.g. in a longitudinal direction or axis of the apparatus. In an embodiment, the expandable members may be spatially distal along an axis, e.g. a longitudinal axis, of the housing and/or opening. The expandable members may share a common axis, e.g. longitudinal axis, with the housing and/or opening.

The expandable members may be provided adjacent to each other or in proximity to each other. In an embodiment, the expandable members may be separated by a gap sufficient to allow actuation, e.g. inflation, of each expandable member without interference with another expandable member.

The housing may be rigid or flexible.

In an embodiment, the housing may be flexible, e.g. may be provided in the form of a flexible cuff or sleeve, which may for example be made of fabric, canvas, or the like. By such provision, the apparatus may be conveniently stored when not in use, and may be conveniently placed on a subject's limb when required. The housing may be made from a durable and/or hygienic material, e.g. from a polymeric material such as a PVC-coated polyester.

The expandable members may form part of the housing. The housing may contain or may define the expandable members.

The expandable members may have a stowed configuration and a deployed configuration. In use, in a stowed configuration, the expandable members may not engage the limb, may not apply pressure on the limb, and/or may not apply sufficient pressure on the limb so as to restrict blood flow. In a deployed configuration, the expandable members may be configured to engage the limb, to apply pressure on the limb, and/or to apply sufficient pressure on the limb so as to restrict blood flow.

One or more expandable members, typically each expandable member, may comprise or may be provided in the form of an inflatable member, such as an inflatable bladder. Thus, in a stowed configuration, the bladder(s) may be deflated, and in a deployed configuration, the bladder(s) may be inflated. In such instance the limb-compressing apparatus may be defined as a pneumatic tourniquet.

The expandable members may be made from a material suitable to undergo repeated cycles of expansion and contraction, e.g. inflation and deflation. The expandable members may be made from a polymeric material such as a PVC-coated polyester.

Advantageously, each expandable member, e.g. bladder, may be actuated, e.g. inflated, independently.

Advantageously, in use, upon actuation of the apparatus, a first expandable member may be actuated, e.g., a first bladder may be inflated. The apparatus may be configured to allow actuation of a second expandable member, for example after a predetermined amount of time. By such provision, when a second expandable member is actuated from its stowed configuration to its deployed configuration so as to compress a subject's limb, the first expandable member may be disengaged from the subject's limb, e.g. may be moved from its deployed configuration to its stowed configuration. Advantageously, this allows the apparatus to maintain the desired level of compression on the limb and/or the desired level of blood flow restriction, while reducing the likelihood of local ischaemia and/or mechanical vascular and nerve damage at the point of application of the apparatus.

The apparatus may comprise fastening means for securing the apparatus, e.g. housing thereof, on a subject's limb. The fastening means may comprise conventional fastening mechanisms such as buttons, rivets, hook and loop fasteners (e.g., Velcro®) or the like.

Conveniently, the Velcro® fasteners may be provided on a surface of the housing, e.g., on a surface of the cuff or sleeve. Typically, one or more Velcro® strips (e.g. loops) may be provided on an outer surface of the cuff or sleeve, and a Velcro fastener (e.g. hooks) may be provided on a fastening portion of the apparatus typically located near at or near an end of the apparatus. By such provision, in use, the apparatus may be placed on a subject's limb so as to engage the limb, the fastening portion may be wrapped over a portion of the apparatus, e.g. of the housing, so as to bring the Velcro® fastening portion in contact with the one or more Velcro® strips, thus securing the apparatus on the subject's limb.

The limb-compressing apparatus may comprise actuating means for actuating the at least two inflatable bladders. The actuating means may be configured to actuate each inflatable bladder independently and/or selectively.

The actuating means may comprise pressure-inflating means, e.g. one or more pumps.

The apparatus may comprise or may be connectable to a plurality of pumps, each pump associated with a respective expandable member, e.g. bladder. Alternatively, the apparatus may comprise or may be connectable to a pump selectively associated with a plurality of expandable members, e.g. bladders. By such provision the apparatus may be actuated using a single pump, thus reducing costs and improving convenience, while allowing a user to selectably inflate each bladder.

One or more actuators, e.g. pumps, may be mechanically powered. In an embodiment, one or more actuators may comprise or may be a mechanical pump, e.g. a foot pump, a hand pump, or the like. By such provision, the tourniquet may be deployed and secured on a subject's limb without the need for external power, e.g. electrical power.

One or more actuators, e.g. pumps, may be externally powered, e.g., may be electrically powered. In such instance there may be provided a power supply, e.g. a portable power supply such as a battery, capable of powering one or more actuators, e.g. pumps and/or any accessory therefor, such as a compressor.

Advantageously, the limb-compressing apparatus, e.g. tourniquet, may be configured to be used in combination with the limb-stabilisation apparatus. By such provision, the entire system may provide not only an improved means of reducing or preventing haemorrhage, and may also prolong tolerance to ischemia in the subject's limb by providing a portable, simple and effective recirculation, oxygenation, and/or temperature control (e.g. cooling) apparatus, thus improving the chance of full or partial recovery to the subject's limb.

The limb-stabilisation apparatus may be attached or attachable to the limb-compressing apparatus, e.g. by Velcro®, clips, straps, rivets, etc.

The limb-stabilisation apparatus and the limb-compressing apparatus may be provided as a combined or integrated system.

Advantageously, the apparatus according to the present invention permits effective recirculation of the blood lost from a wound in a subject's limb, while avoiding unnecessary surgical intervention at the point of injury (e.g., to close one or more wounds), which may help transport the subject more quickly to a medical destination (e.g. a hospital) while maintaining the injured limb's metabolic needs.

According to a second aspect of the present invention there is provided a method of treating a subject's limb, comprising:

placing a subject's limb in a limb-receiving container arranged to receive a portion of the limb and to collect blood exited from the limb; and

recirculating said blood from the limb-receiving container to one or more regions of the limb.

The method may comprise cannulating an arterial side of the limb to a blood recirculation system configured to recirculate blood collected in the limb-receiving container to one or more regions of the limb.

The method may comprise determining a desired pressure for the blood recirculation system.

The method may comprise activating the blood recirculation system so as to recirculate blood at the desired pressure.

The method may comprise monitoring the pressure and/or flow in the blood recirculation system.

The method may comprise regulating and/or controlling blood flow and/or pressure within the blood recirculation system. The method may comprise regulating and/or controlling a/the pump. For example, if the blood pressure within the pressure blood recirculation system falls below a predetermined or desired value, the method may comprise controlling a/the pump power, so as to reach the predetermined or desired pressure.

The method may comprise monitoring the blood levels in the sump.

When the blood level in the sump is below a predetermined level, the method may comprise one or more of the following:
(a) Stopping blood recirculation, for example by shutting down a/the associated pump, e.g. until blood level in the pump reaches a/the predetermined level; and
(b) Supplying external blood into the recirculation system.

By such provision, the method may ensure that, in the event that there is insufficient blood level/volume in the sump to provide the desired blood pressure and/or blood flow, the recirculation system does not cause air to be pumped into the subject.

The method may comprise automatically regulating and/or controlling blood flow and/or pressure within the blood recirculation system, e.g. by controlling the/a pump.

The method may comprise automatically taking action based on the level or volume of blood in the sump. For example, if the blood level or volume in the sump is below a predetermined level, the method may comprise automatically performing one or more of the following:
(c) Stopping blood recirculation, for example by shutting down a/the associated pump, e.g. until blood level in the pump reaches a/the predetermined level; and
(d) Supplying external blood into the recirculation system.

The method may comprise applying a limb-compressing apparatus on a region of the limb so as to reduce, restrict or prevent blood flow.

The features described in respect of the apparatus according to a first aspect may apply equally in respect of the method according the second aspect, and are not repeated here for brevity.

### BRIEF DESCRIPTION OF DRAWINGS

Embodiments of the invention will now be given by way of example only, and with reference to the accompanying drawings, which are:

FIG. 1 a perspective view from above of a limb-stabilisation apparatus according to a first embodiment of the present invention;

FIG. 2 a perspective view from above of the limb-stabilisation apparatus of FIG. 1, in use;

FIG. 3 a perspective view from above of a limb-compressing apparatus for use with the limb-stabilisation apparatus of FIG. 1;

FIG. 4 a perspective view from above of another embodiment of a limb-compressing apparatus for use with the limb-stabilisation apparatus of FIG. 1;

FIG. 5 the apparatus of FIG. 4, in a deployed or “in use” configuration;

FIG. 6 a schematic view from above of a cuff of the apparatus of FIG. 4;

FIG. 7 a schematic side view of the sump of the apparatus of FIGS. 1 and 2.

### DETAILED DESCRIPTION OF DRAWINGS

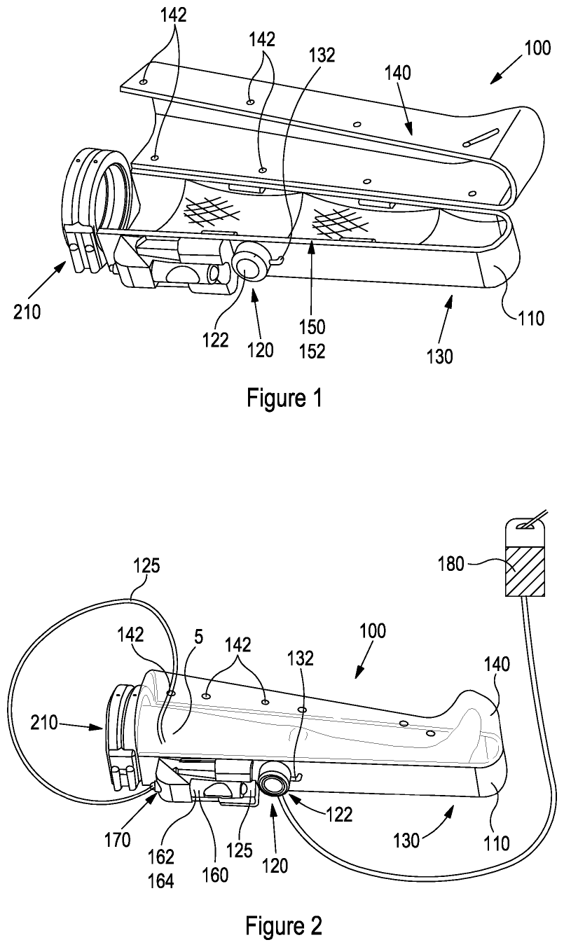

Referring to FIGS. 1 and 2 there are shown a perspective view from above of a limb-stabilisation apparatus, generally designated 100, according to a first embodiment of the present invention.

The limb-stabilisation apparatus 100 has a limb-receiving container 110 arranged to receive a portion of the limb 5, and to collect blood exited from the limb 5. The limb-stabilisation apparatus 100 includes a blood recirculation system 120 configured to recirculate blood from the limb-receiving container 110 to one or more regions of the limb 5.

Advantageously, the limb-stabilisation apparatus 100 is associated with and is located below a tourniquet 210 configured to reduce, restrict or prevent blood flow in the region of the tourniquet 210. This may assist in isolating the injured limb from the subject's circulatory system, and may help prevent or reduce bleeding and/or extremity haemorrhage exsanguination.

The limb-receiving container 110 is configured to contain blood. By such provision, the limb-receiving container 110 is capable of containing the blood exited/lost from the injured limb 5, thus ensuring that all the blood lost by the subject may be recirculated. The limb-receiving container 110 is rigid and is impermeable to liquids, e.g. blood.

The limb-receiving container 5 includes a sump 130 (as best shown in FIG. 7) for collecting blood exited from the limb and/or contained in the limb-receiving container 110.

The apparatus 100, and in this embodiment the blood recirculation system 120, includes a pump 122 for pumping and/or circulating the blood collected in the sump 130.

The apparatus 100 has comprise a sump connector 132 which provides fluid communication between the sump 130 and the pump 122.

Advantageously, the pump 122 is capable of recirculating blood to one or more regions of the limb 5. In the embodiment of FIG. 2, the pump 122 is in fluid communication with a cannula 125 which is connected to an arterial vessel of the limb 5. By such provision, the apparatus 100 can recirculate the blood lost by the limb 5 and collected into the sump 130 back into an arterial system of the limb 5, thus maintaining blood flow through the limb 5 in a simple and effective manner, for example prior to, or after, surgery.

In other embodiments, there may be provided a plurality of cannulations, each cannulation being connected to an arterial vessel upstream from an associated wound, e.g., upstream from a wound from which blood exits the vessel.

The apparatus 100 includes a lid 140. In this embodiment, the lid 140 is hingedly connected to the container 110 and can be opened and closed by a user. This may allow, in use, protection of the limb 5 and/or may provide a hygienic and/or sealed environment for a/the injured limb 5.

While the embodiment of FIGS. 1 and 2 shows a single lid 140, it will be appreciated at the lid 140 may be provided as a plurality of adjacent lids which can be opened and closed independently, thus allowing exposure of a limited region of the limb 5.

The lid 140 has a plurality of openings 142 in the form of holes configured to receive a medical tube such as a cannula 125, which openings 142 are provided at different positions in the lid 140. By such provision, a/the cannula 125 may be easily connected to a vessel, e.g. an arterial vessel, on the limb 5 at a location near a particular opening 142, in use.

Advantageously, the lid 140 is made of a transparent material. By such provision, a used may be able to connect a cannula 125 to an arterial vessel and/or may be able to monitor the apparatus 100 and/or the subject's limb 5, in use.

In this embodiment, the container 110 is opaque. However, it other embodiment, the container may also be transparent.

As best shown in FIG. 1, the container 100 includes a limb-suspension member 150 configured to suspend the limb 5, in use, from the bottom of the container 110. Advantageously, the limb-suspension member 150 allows the limb 5 to be supported within the container 110, while ensuring the limb 5 does not rest on or does not contact a substantial area of the container 110. This may provide a more hygienic environment, allowing the blood to exit vessels and/or wounds in the limb 5 and be collected in the sump 130 without significant or prolonged contact with an outer surface of the limb 5, e.g. skin thereof, thus reducing the risks of infection.

In this embodiment, the limb-suspension member 150 is provided in the form of a net or mesh 152. A net of mesh may provide adequate support to the limb 5, while helping blood drainage and/or collection into the container 110, e.g. blood sump 130.

The apparatus 100 also includes a blood oxygenating device 160 and a blood temperature control device 162. In this embodiment, a combined and/or integrated device 164 is provided which functions as a blood oxygenating device 160 and a temperature control device 162. By such provision, the device 164 may provide not only blood recirculation to the injured limb 5, but may also provide blood oxygenation thus providing the metabolic needs of the limb 5. The provision of a temperature control device 162, e.g. a cooling device, capability may also help reduce the oxygen requirements of the limb 5.

The limb-stabilisation apparatus 100 also includes a blood-treatment unit 170. The blood treatment unit 170 is configured to alter, e.g. to remove, certain components of the blood, and in this embodiment to filter one or more components of the blood that are susceptible of inducing or suppressing an inflammatory response in the subject such as cytokines.

As best shown in FIG. 7, the limb-receiving container 110 includes a sump 130 for collecting blood exited from the limb and/or contained in the limb-receiving container 110.

The blood sump 130 is located near a lower end of the limb-receiving container 110. In use, the limb-receiving container 110 is inclined such that the blood lost from the limb 5 flows to and/or is collected into the sump 130.

The blood sump 130 is provided with blood measuring means 131 for measuring the level and/or volume of blood in the sump 130. In this embodiment, the blood measuring means 131 are provided in the form of graduations 133, and also include sensor 134 configured to measure the level and/or volume of blood in the sump 130.

The apparatus includes a blood pressure sensor 125 for measuring the pressure within the blood recirculation system 120.

Advantageously, if the blood pressure within the pressure blood recirculation system 120 falls below a predetermined or desired value, the power of the pump 120 may be altered, for example in order to reach a predetermined or desired pressure.

If the blood level and/or volume in the sump 130 falls below a predetermined level, blood recirculation may be stopped, for example by shutting down the pump 122, e.g., until blood level in the sump 130 reaches a/the predetermined level

In this embodiment, as best shown in FIG. 2 there is also provided an external blood supply in the form of a blood bag 180. Advantageously, if the blood level and/or volume in the sump 130 falls below a predetermined level, external blood from the blood bag 180 can be fed into the recirculation system 120.

By such provision, the apparatus/system 100 ensures that, in the event that there are insufficient blood levels in the sump 130 to provide the desired blood pressure and/or blood flow, the recirculation system 120 does not cause air to be pumped into the subject. Advantageously, if the blood level and/or volume in the sump 130 falls below a predetermined level, the apparatus 100 automatically pumps external blood from the blood bag 180 into the recirculation system 120.

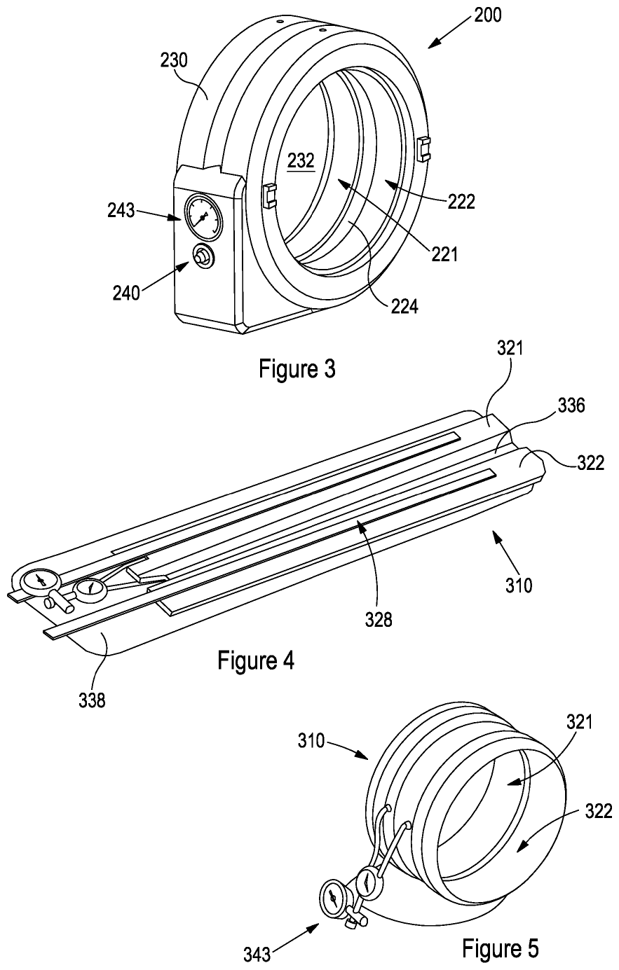

Referring now to FIG. 3, there is shown a perspective view from above of a limb-compressing apparatus 210 for use with the limb-stabilisation apparatus 100 of FIG. 1. The limb-compressing apparatus 210 is a dual tourniquet 210.

The tourniquet 210 has a pair of inflatable bladders 221,222, partially located within a housing 230. In this embodiment, the housing 230 is a rigid housing 230.

The housing defines an opening 232 for receiving a limb of a subject, e.g., a portion of an arm or a leg.

The bladders 221,222 are spatially arranged in a longitudinal direction along a axis of the housing 230 and opening 232. The bladders 221,222 share a common axis with the housing 230 and the opening 232.

The bladders 221,222 are provided adjacent to each other. In this embodiment, the bladders 221,222 are separated by a gap 224 having a size sufficient to allow inflation of each bladder without interference with the other bladder.

The housing 230 and/or opening 232 thereof is/are dimensioned so as to be capable of receiving a portion of a subject's limb. As such, the apparatus 210 is capable of receiving a subject's limb having a range of sizes, e.g., diameter. For example, the apparatus may be manufactured in a range of sizes, each size being designed to receive a predetermined range of limb sizes, for example corresponding to a typical region of a limb, e.g., a forearm, an arm, a lower leg (calf), or an upper leg (thigh).

The apparatus 210 has a gas inlet 240 configured to be connected to a pump (not shown) so as to independently and selectively inflate each bladder 221,222. The apparatus also has a pressure gauge or manometer 243 to monitor the pressure in the bladders.

Referring now to FIGS. 4 and 5, there is shown a tourniquet apparatus 310 according to a second embodiment of the present invention. The tourniquet 310 of FIG. 4 is similar to the tourniquet 210 of FIG. 3, like part denoted by like numerals, incremented by ‘100’.

The tourniquet 310 also has a housing 330 and a pair of bladders 321,322. However, in this embodiment, the housing 330 is a flexible housing 330. The housing 330 has an inflatable elongate portion 336 which has been heat-sealed along a length thereof in its central region so as to define two separate inflatable bladders 321,322. The inflatable bladders 321,322 are made a PVC-coated polyester material which allows the tourniquet 310 to undergo repeated cycles of expansion and contraction, e.g. inflation and deflation, without mechanical damage.

The tourniquet 310 also has a non-inflatable cuff portion 338 at an upper end of the bladders, arranged to affix and secure the tourniquet 310 on a subject's limb, as will be explained later in more detail.

The tourniquet 310 has Velcro® fasteners 328 to secure the tourniquet on a subject's limb.

FIG. 4 shows the tourniquet 310 in a non-inflated configuration, when not in use. FIG. 5 shows the tourniquet 310 in a deployed or “in use” inflated configuration. For illustration purposes, both inflatable bladders 321,322 are shown as inflated. However, the tourniquet 310 allows each bladder 321,322 to be inflated separately and/or independently, as will be explained later in more detail.

A manometer 343 is provided to measure pressure in the bladders 321,322.

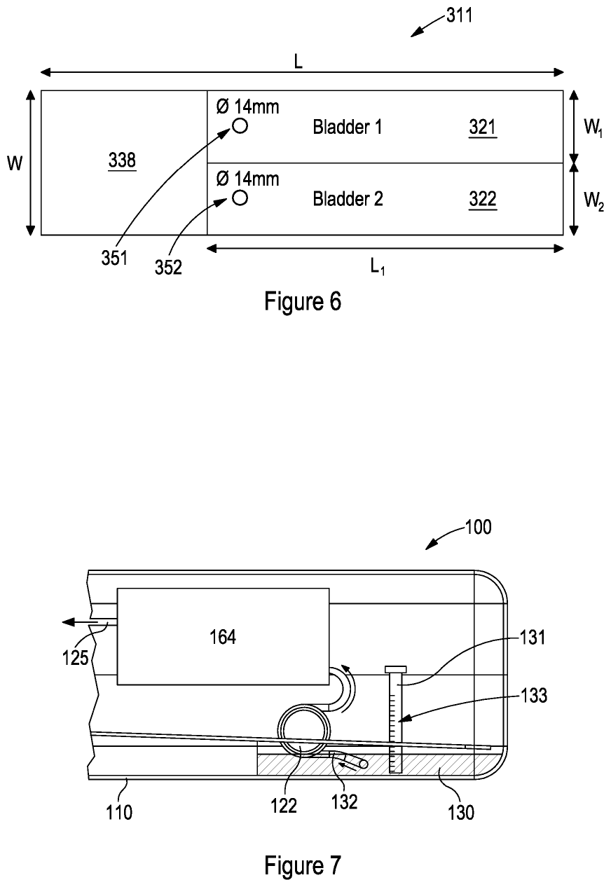

FIG. 6 shows a schematic view from above of a cuff structure 311 of the tourniquet 310 of FIG. 4. The tourniquet 310 has a flexible housing 330 provided in the form of a cuff 311. The tourniquet 310 has an inflatable elongate portion 336 which has been heat-sealed along a length thereof in its central region so as to define two separate inflatable bladders 321,322. The tourniquet 310 also has a non-inflatable cuff portion 338 at an upper end of the bladders, arranged to affix and secure the tourniquet 310 on a subject's limb.

In this embodiment, the dimensions of the cuff 311 are selected to ensure that the tourniquet 310 is suitable for upper and lower limbs. As such, the cuff 310 was designed in accordance with dimensions stated by McDowell and colleagues in a 2008 National Statistics Report on Anthropometric Reference Data for Children and Adults in the United States between 2003-2006 (http://www.cdc.gov/nchs/data/nhsr/nhsr010.pdf), where the average adult mid-arm and mid-thigh circumferences were recorded as 39.4 cm and 55 cm, respectively.

Thus, in this embodiment, the cuff 311 had a total length L of 850 mm, and a total width W of 140 mm. The length L1 of the bladders was 600 mm, and the width W1,W2 of each bladder was 70 mm. The diameter d1,d2 of each port 151,152 for connecting to a pump inlet such as an inlet tube (not shown) was 14 mm.

It will be appreciated that the embodiments of the invention hereinbefore described are given by way of example only and are not meant to limit the scope thereof in any way.

1. A limb-stabilisation apparatus comprising:
a limb-receiving container arranged to receive a portion of the limb and to collect blood exited from the limb; and
a blood recirculation system configured to recirculate blood from the limb-receiving container to one or more regions of the limb.

2. A limb-stabilisation apparatus according to claim 1, further comprising a limb-compressing apparatus configured to reduce, restrict or prevent blood flow in the region of the limb-compressing apparatus.

3. A limb-stabilisation apparatus according to claim 1, wherein the limb-receiving container is configured to receive a portion of the limb below the limb-compressing apparatus.

4. (canceled)

5. A limb-stabilisation apparatus according to claim 1, wherein the limb-receiving container comprises and/or defines a sump for collecting the blood exited from the limb and/or contained in the limb-receiving container.

6. A limb-stabilisation apparatus according to claim 5, wherein the blood recirculation system comprises a pump for pumping and/or circulating the blood, and wherein the pump is in fluid communication with the sump.

7. (canceled)

8. A limb-stabilisation apparatus according to claim 6, wherein the pump is capable of recirculating the blood to one or more regions of the limb.

9. A limb-stabilisation apparatus according to claim 6, wherein the pump is connectable to one or more cannulae for connection to a region of the limb.

10. (canceled)

11. A limb-stabilisation apparatus according to claim 6, wherein the apparatus comprises a cannulation router configured to guide pumped blood to one or more cannulae, wherein the cannulation router has at least one inlet, and a plurality of outlets, each outlet having an open and a closed position and being connectable to a respective cannula.

12.-15. (canceled)

16. A limb-stabilisation apparatus according to claim 1, wherein the limb receiving container comprises or is provided with a limb-suspension member configured to suspend the limb, in use.

17.-18. (canceled)

19. A limb-stabilisation apparatus according to claim 5, wherein the limb-stabilisation apparatus is configured to measure a level and/or volume of blood in the sump.

20. A limb-stabilisation apparatus according to claim 1, wherein the apparatus comprises, at least one blood pressure sensor for measuring pressure within the blood recirculation system.

21. A limb-stabilisation apparatus according to claim 6, wherein the pump may be is controllable and/or adjustable based on a measured blood pressure and/or blood level.

22. A limb-stabilisation apparatus according to claim 5, further comprising an external blood container configured to supply or feed external blood into the recirculation system if blood level and/or volume in the sump falls below a predetermined level.

23.-24. (canceled)

25. A method of treating a subject's limb, comprising:
placing a subject's limb in a limb-receiving container arranged to receive a portion of the limb and to collect blood exited from the limb; and
recirculating said blood from the limb-receiving container to one or more regions of the limb.

26. A method according to claim 25, comprising cannulating an arterial side of the limb to a blood recirculation system configured to recirculate blood collected in the limb-receiving container to one or more regions of the limb.

27. A method according to claim 26, comprising determining a desired pressure for the blood recirculation system, and activating the blood recirculation system so as to recirculate blood at the desired pressure.

28. (canceled)

29. A method according to claim 26, comprising monitoring the pressure and/or flow in the blood recirculation system, and/or comprising regulating and/or controlling blood flow and/or pressure within the blood recirculation system.

30. (canceled)

31. A method according to claim 25, comprising monitoring the blood levels in a sump.

32. A method according to claim 31, wherein, when the blood level in the sump is below a predetermined level, the method comprises one or more of the following:
(a) stopping blood recirculation until blood level reaches the predetermined level; or
(b) supplying external blood into the recirculation system.

33. A method according to claim 25, further comprising applying a limb-compressing apparatus on a region of the limb so as to reduce, restrict or prevent blood flow.
